(19) United States
(12) Patent Application Publication
(10) Pub. No.: US 2015/0165230 A1
(43) Pub. Date: Jun. 18, 2015

(54) Vision Stimulator

(71) Applicant: Daniel D. Gottlieb, Sandy Springs, GA (US)

(72) Inventor: Daniel D. Gottlieb, Sandy Springs, GA (US)

(21) Appl. No.: 14/576,131

(22) Filed: Dec. 18, 2014

Related U.S. Application Data
(60) Provisional application No. 61/917,804, filed on Dec. 18, 2013.

(51) Int. Cl. A61N 5/06

(52) U.S. Cl. CPC A61N 5/0622; A61N 2005/0629; A61N 2005/0651

(57) ABSTRACT

A handheld device is disclosed for assisting in stimulating vision improvement in individuals with visual field loss caused by brain trauma or cortical visual impairment. The device comprises a number of lights arranged in a pattern, which blink sequentially to provide the visual effect of a single light moving across the pattern. The device may be set to create the visual motion effect, and then placed at the edge of the field of vision loss so that the individual's brain will sense movement within the visual field and track that movement into the blind spot. The device includes controls to adjust the brightness and frequency of the blinking lights.

### PRIORITY CLAIM

This application claims the benefit of U.S. Provisional Patent Application No. 61/917,804 filed by this same inventor on Dec. 18, 2013.

### TECHNICAL FIELD

The present invention relates generally to a device for assisting in stimulating vision improvement in individuals suffering from vision field loss, and, more specifically, to a device comprising a series of lights that blink sequentially to simulate movement and assist a patient's brain in expanding the perceived field of vision.

### BACKGROUND OF THE INVENTION

Many people suffer from a loss of their normal field of vision, or hemianopia, created by some type of brain trauma such as an accident or stroke. This brain trauma may cause an individual's brain to stop perceiving light from an area of the field of vision, even though the individual's eyes are still physically capable of receiving light from the entire visual field. The limited field of vision is not only dangerous, but can create psychological issues for the individual that interfere with recovery from the trauma.

Over the years it has been found that providing a rapidly moving light in and around the individual's field of vision loss may help stimulate the brain into sensing the presence of the light and thereby expand the area from which the brain will process the presence of light, i.e., expand the narrowed field of vision. The rapidly moving light has most commonly been provided by having a physician physically move a light emitting source, such as a pen light, around the boundaries of the individual's field of vision loss. A pen light may be shaken back and forth and/or in circular patterns to provide the visual effect of rapid motion and slowly stimulate the brain to sense the motion as it moves from the patient's field of perception into the field of visual loss.

The problem with shaking a pen light is that the motion is inconsistent and difficult to adjust to a speed and brightness that may be more beneficial to the patient. Also, the process of shaking the pen light can often be uncomfortable for the physician or caregiver (the present inventor has developed carpal tunnel syndrome due, in part, to the practice of shaking a pen light for patients). If the physician or caregiver experiences pain or tiredness from shaking a light, then they may be less inclined to continue the therapy for the desired time period, or to move the light around and change the motion to experiment with positions that are most effective in improving the patient's field of vision.

To date, no devices have been developed that effectively simulate this known light treatment therapy. A device useful for this therapy would need to have adjustability features that allow for a variety of light movement to stimulate various areas of vision loss. Thus there is a need for a device that will emit rapidly moving light that may be used to stimulate an individual's brain that does not have to be moved manually to be effective, and which has adjustability that provides for varying speeds, brightness, and/or patterns of light movement.

### SUMMARY OF THE INVENTION

The present invention provides a handheld device that simulates the visual effect of moving a pen light. In one embodiment of the present invention the handheld device comprises multiple lights arranged in a pattern such that when the lights are turned on and off sequentially, the emitted light will provide a visual effect that simulates movement of a single light across the light pattern, and further comprises means for controlling the speed and brightness of the sequential illumination of the lights.

In another embodiment, the lights of the handheld device are LEDs. The LEDs may be spaced in a substantially straight line, or arranged in another pattern, such as a circle or a sine wave.

In yet another embodiment of the invention, the lights comprise LEDs that are spaced about 1/16 inches apart. The spacing of the lights may be somewhat imprecise to provide a slightly more natural visual effect.

In still another embodiment of the invention, the lights of the device comprise LEDs that each blink in a frequency that falls within the range of about 0.5 to about 50 Hz.

For another embodiment, the LEDs of the device are arranged in a circular or sine wave pattern to provide simulated motion in different directions.

The present invention further comprises a method for treating individuals having a field of vision loss caused by visual neglect or cortical visual impairment. The method comprises providing a handheld device that simulates the visual effect of moving a pen light. The handheld device may comprise multiple lights arranged in a pattern such that when the lights are turned on and off sequentially, the emitted light will provide a visual effect that simulates movement of a single light across the light pattern, and further comprises means for controlling the speed and brightness of the sequential illumination of the lights. The method further comprises turning on the device so that the lights begin blinking, and then placing the device at about the edge of the individual's field of vision loss.

In yet another embodiment of the present invention, the treatment method further comprises moving the device to various locations in the individual's field of vision to provide different types of visual stimulation. The method may further comprise adjusting the brightness of the LEDs and the frequency of the blinking to alter the visual stimulation.

### BRIEF DESCRIPTION OF THE DRAWINGS

Drawings and photographs are included herein that illustrate one or more embodiment(s) of the present invention. It is to be noted the figures presented are intended solely for the purpose of illustration and that they are, therefore, neither desired nor intended to limit the invention to any or all of the exact details of construction shown, except insofar as they may be deemed essential to the claimed invention.

FIG. 1 is a perspective view of a device that is one embodiment of the present invention.

FIG. 2 is a rear panel of the device of FIG. 1.

FIG. 3 is a schematic diagram of the electronics for the device of FIG. 1.

FIG. 4 shows an alternative embodiment of the present invention.

FIG. 5 shows an alternative embodiment of the present invention.

FIG. 6 shows an alternative embodiment of the present invention.

### DETAILED DESCRIPTION OF THE INVENTION

Accordingly, the present invention provides a device 10 that emits light in a manner that will simulate rapid movement of a single light, and assist in stimulating visual improvement within a perceived field of vision loss. To simulate movement, the present invention may utilize a series of lights 20a-20f arranged substantially linearly, or in another sequential pattern. Lights 20 may be designed to blink on and off sequentially so that a single point of light appears to be moving across the light series 20a-20f. The size and design of device 10 is virtually unlimited, but the size of the lights should be small enough to be placed along the boundary of the patient's field of vision loss. If the lights are too large, then the effect of motion may be lost, or the brain not be sufficiently stimulated to expand the vision field.

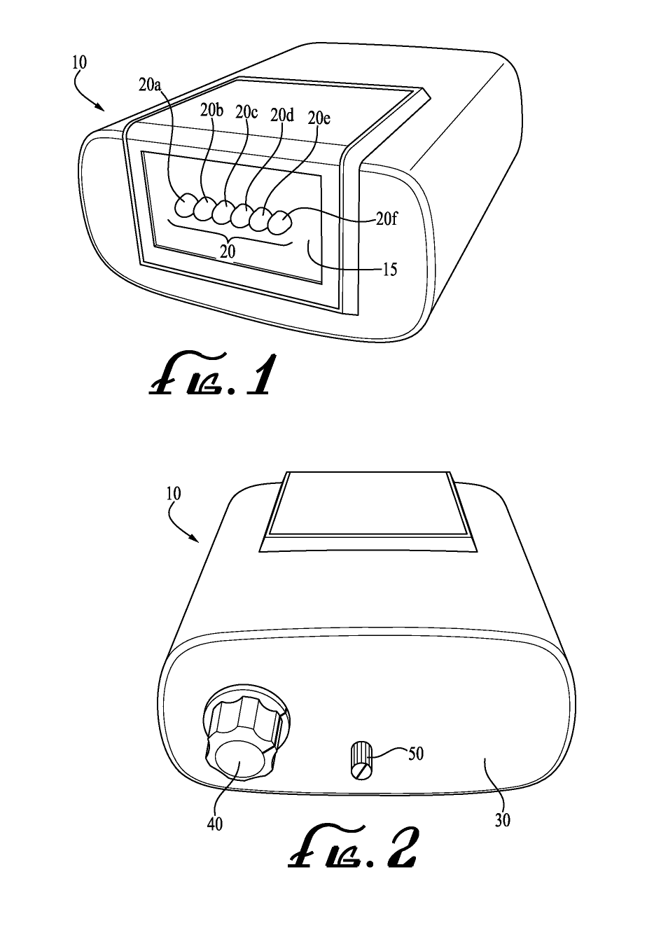

Turning now to FIG. 1, device 10 embodying the present invention is shown. Device 10 may comprise a front panel 15 having six (6) light emitting diodes (LEDs) 20a-20f on the front panel. When these LEDs 20a-20f are turned on and off in rapid succession, the visual effect is preferably of a single light moving along the row of the LEDs 20a-20f. By “rapid succession” it is meant that the light pattern is completed in a manner that approximates a human wagging a pen light. It has been found that, with a device having about 6 lights, cycles in the range of about 0.5 to about 50 Hz are useful, but there is no limit to the speed of the successive blinking of the lights.

This simulated motion is very similar to the visual effect of shaking a light pen having a single point of light as currently used to stimulate a patient's vision. In practice, device 10 may be placed along the edges of the field of vision loss so that the patient's brain may perceive the light motion and begin to sense the ability to expand the perceived visual field. Device 10 may be moved around and held in various positions and, with the help of patient feedback, the most effective positions for treatment may be found.

For example, an effective position is typically right at the edge of the patient's field of vision loss, so that some of the lights are within the patient's current vision and other lights are within the patient's blind spot. The appearance of light movement may stimulate the patient's brain to perceive the lights that are blinking with in the field of vision loss, thereby narrowing the field. Once an effective location for device 10 is identified, device 10 may be held or secured in the desired location for a brief period while the patient attempts to perceive light motion within the field of vision loss. The brightness and/or blinking frequency of the lights may be adjusted to assist with stimulating the brain to broaden the field of vision. After a brief time, the device may be moved to another location near the edge of the field of vision loss.

Use of this device has been successful in treating patients with cortical visual impairment (“CVI”), including infants born with CVI or people who have developed visual impairment through some type of brain trauma as opposed to injury to the eye. With the device turned on and moved toward the area of field loss, the device operator may get immediate feedback from the patient that their vision is improving, or that they feel some type of ability to perceive motion within their blind spot. Even babies have been seen to follow the device with their eyes, when they previously appeared to have little to no vision.

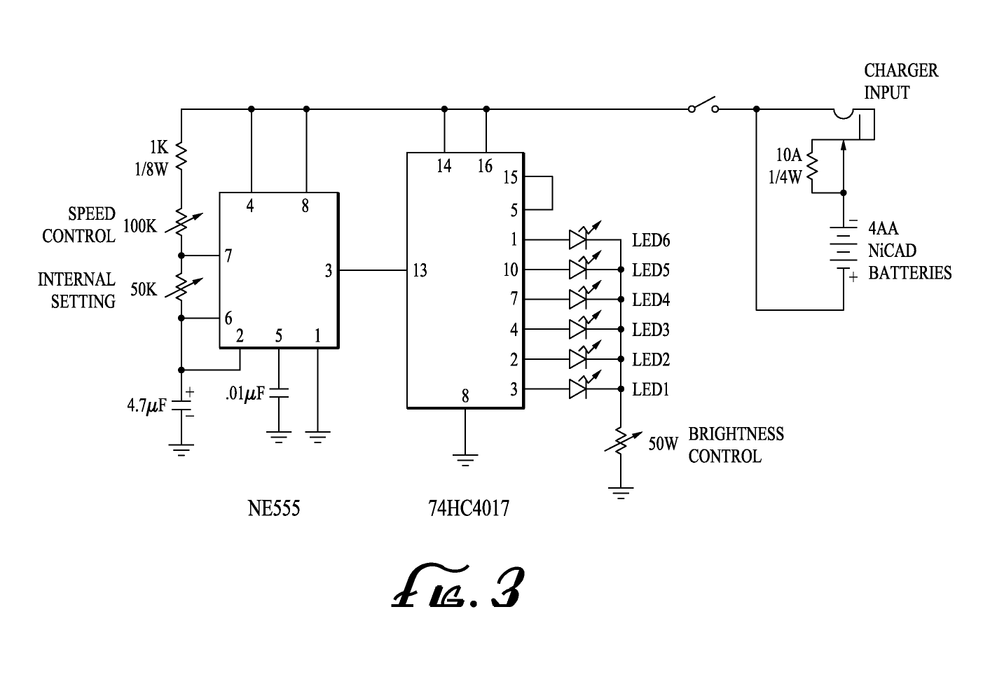

FIG. 2 shows control panel 30 of device 10, shown in the illustration as the back of device 10. In practice, the controls for device 10 may be located anywhere so long as the controls do not interfere with the operation of lights 20. Also, the controls are preferably easy to maneuver even while the device is in use. In the illustrated embodiment, knob 40 serves as an on/off switch for lights 20, and also controls the brightness of lights 20. In practice, the on/off switch and brightness controls could be different switches. Dial 50 controls the frequency of the sequential blinking of the lights 20. These adjustments may be manipulated as needed to best serve a given patient. Different patients may react better to different speeds of simulated movement and/or brightness.

FIG. 3 is a schematic diagram of one possible electronic configuration for device 10 of FIG. 1. This is just one manner of designing the present invention. As noted, any manner of providing a series of lights that blink sequentially to simulate movement within a handheld device is intended to be within the scope of the present invention.

Device 10 is illustrated in FIG. 1 having 6 LEDs 20a-20f in a straight line, spaced approximately 1/16 inches apart. The present invention is in no way intended to be limited to such a light display. More lights with different spacing may easily be utilized, although the present inventor has found a devices with 1/16 inch spacing or less between the LEDs 20a-20f has been the most effective in treating patients. Positive results have also been obtained with devices having 9 LEDs in a substantially straight line.

Surprisingly, devices that have imprecise spacing between LEDs 20a-20f have been found to be more effective than prototype devices having consistent spacing between the lights. By “imprecise spacing”, it is meant that the spacing between each of lights 20a-20f are inconsistent, but that each is less than 1/16 inches. Further, devices in which the lights 20a-20f are not in a perfectly straight line have proven to be more successful in treating patients than devices with more exacting specifications. The present inventor has not conducted enough experimentation to determine whether imprecise spacing and imperfect linear positioning of lights 20 has a direct effect on the efficacy of the device for treatment, but to date patients seem to prefer the devices built with slight imperfections. It is theorized the devices with imperfections provide a better simulation of the natural movement previously obtained by moving a light pen, and that perhaps the digital precision of evenly spaced lights in a perfectly straight line are less effective at stimulating the brain.

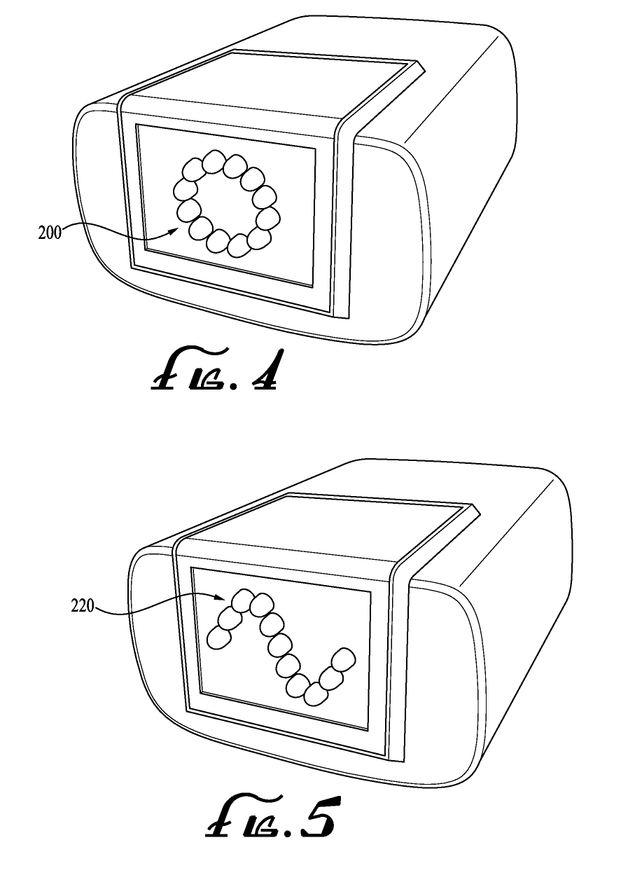

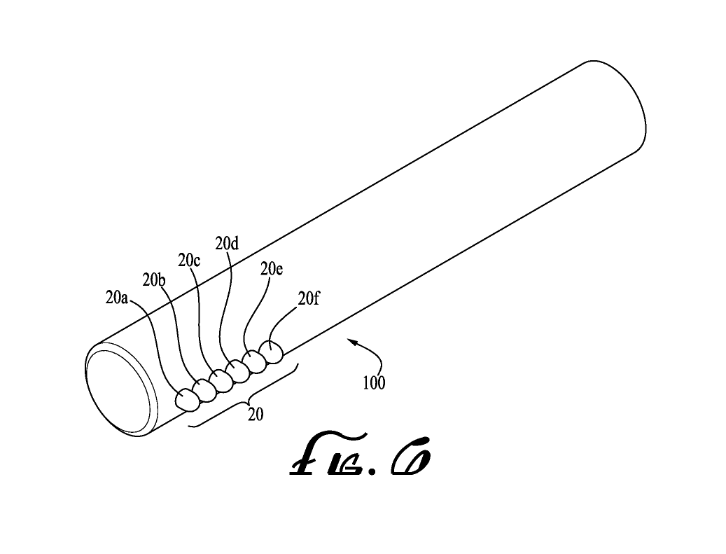

A device with a large number of lights 20 might not be limited to straight line movement, as different patterns of movement including circles or “S”-patterns may be useful to stimulate brain function. FIG. 4 shows a device 10 having lights 200 arranged in a circular pattern. In the embodiment of FIG. 4, the rapid, sequential blinking of the lights is intended to simulate moving a light pen in a circle. FIG. 5 shows a device 10 having lights 220 arranged in an “S” pattern, or sine wave. The embodiment set forth in FIG. 5 may be designed so that the rapid, successive blinking of the lights simulates moving a light in an “S” Pattern.

In addition, the device could be designed such that the pattern of light illumination changes, in order to simulate various movement. For example, if the lights are arranged in a straight line, the light illumination sequence could vary from left to right and then right to left. If the lights are arranged in a circle, the illumination pattern may vary from clockwise to counter-clockwise. In more complex light patterns, the sequence may vary in other directions as well. A main consideration for the light illumination patterns is that the visual effect still simulates movement of a single light in a pattern. If multiple illumination patterns are desired, a separate control on the device may be included to assist in switching between patterns.

Device 10 is also not intended to be limited to the box shape shown in FIGS. 1-5. In practice, a smaller device may be more desirable, and it is believed any device that fits comfortably in the users hand would be preferred. FIG. 6 shows an embodiment of the present invention encompassed in a pen shape device 100. It is envisioned that the device may one day be a size and shape similar to a pen that simply needs to be held up in the appropriate spot for the patient.

Having thus described exemplary embodiments of the present invention, it should be noted by those skilled in the art that the disclosures herein are exemplary only and various other alternatives, adaptations, and modifications may be made within the scope and spirit of the present invention. Accordingly, the present invention is not limited to the specific embodiments as illustrated herein, but is only limited by the following claims.

I claim:

1. A handheld device for stimulating vision within the field of vision loss for an individual suffering from visual neglect comprising:
multiple lights arranged in a pattern such that when the lights are turned on and off sequentially, the emitted light will provide a visual effect that simulates movement of a single light across the light pattern; and
means for controlling the speed and brightness of the sequential illumination of the lights.

2. The device of claim 1 wherein the lights are LEDs.

3. The device of claim 2 wherein the LEDs are spaced in a substantially straight line.

4. The device of claim 3 wherein the LEDs are spaced about 1/16 inches apart.

5. The device of claim 3 wherein the device has at least about 6 LEDs.

6. The device of claim 5 wherein the frequency of the lights blinking is within the range of about 0.5 Hz to about 50 Hz.

7. The method of claim 1 wherein the pattern is selected from the group consisting of a substantially straight line, a circle, and a sine wave.

8. The method of claim 1 wherein the pattern is a substantially straight line.

9. A method for treating an individual having a field of vision loss caused by visual neglect comprising:
(a) providing a handheld device comprising:
(i) multiple lights arranged in a pattern such that when the lights are turned on and off sequentially, the emitted light will provide a visual effect that simulates movement of a single light across the light pattern; and
(ii) means for controlling the speed and brightness of the sequential illumination of the lights;
(b) turning on the device so that the lights of the device are blinking; and
(c) placing the device at about the edge of the individual's visual field loss.

10. The method of claim 9 wherein the device is moved to various positions around the edge of the individual's visual field loss to stimulate vision within the field of loss.

11. The method of claim 9 wherein the speed of the sequential illumination of the lights is adjusted based upon feedback received from the individual.

12. The method of claim 9 wherein the brightness of the illumination of the lights is adjusted based upon feedback received from the individual.

13. The method of claim 9 wherein the lights of the device are LEDs.

14. The method of claim 13 wherein the LEDs are positioned in a substantially straight line.

15. The method of claim 14 wherein the LEDs are spaced about 1/16 inches apart.

16. The method of claim 14 wherein the device has at least about 6 LEDs.

17. The method of claim 16 wherein the frequency of the lights blinking is within the range of about 0.5 Hz to about 50 Hz.

18. The method of claim 13 wherein the LEDs are arranged in a pattern selected from the group consisting of a substantially straight line, a circle, and a sine wave.
